(19) United States
(12) Patent Application Publication
(10) Pub. No.: US 2022/0033872 A1
(43) Pub. Date: Feb. 3, 2022

(54) DEVICE FOR CAPTURING IN SITU AQUATIC MICROBIOMES

(71) Applicants: CIIMAR - CENTRO INTERDISCIPUNAR DE INVESTIGAÇÄO MARINHA E AMBIENTAL, Matosinhos (PT); INSTITUTO SUPERIOR DE ENGENHARIA DO PORTO, Porto (PT)

(72) Inventors: Catarina MAGALHÃES, Matosinhos (PT); Ana Paula MUCHA, Matosinhos (PT); Hugo Manuel DA SILVA RIBEiRO, Matosinhos (PT); Maria Fátima CARVALHO, Matosinhos (PT); Maria Paola TOMASINO, Matosinhos (PT); Marisa ALMEIDA, Matosinhos (PT); Sandra RAMOS, Matosinhos (PT); Alfredo Manuel DE OLIVEIRA MARTINS, Porto (PT); André Miguel PINHEIRO DIAS, Porto (PT); Eduardo Alexandre PEREIRA DA SILVA, Porto (PT); José Miguel SOARES DE ALMEIDA, Porto (PT); Marco MOTA GONÇALVES, Porto (PT); Maurício Miguel DE OLIVEIRA GUEDES, Sao Mamede de Infesta (PT); Nuno Alexandre NETO DIAS, Porto (PT)

(21) Appl. No.: 17/298,428

(22) PCT Filed: Nov. 30, 2019
(86) PCT No.: PCT/IB2019/060370

§ 371 Date: May 28, 2021

(30) Foreign Application Priority Data
Nov. 30, 2018 (PT) 115183

(51) Int. Cl. C12Q 1/24; G01N 1/40; G01N 1/10; G01N 1/14

(52) U.S. Cl. CPC C12Q 1/24; G01N 1/4077; G01N 1/10; G01N 2001/1031; G01N 2001/1418; G01N 2001/4088; G01N 1/14

(57) ABSTRACT

The present disclosure relates to a portable device for collecting and/or concentrating in situ plankton microbiome, configured for submersion in water. The device herein disclosed is a compact and low-cost autonomous biosampler, with the ability to yield DNA samples for later genomic analysis.

### TECHNICAL FIELD

The present disclosure relates to a portable device for collecting and/or concentrating in situ plankton microbiome, configured for submersion in water. The device herein disclosed is a compact and low-cost autonomous biosampler, with the ability to yield DNA samples for later genomic analysis.

### BACKGROUND

Life in aquatic environments, including marine and freshwater ecosystems, is dominated by a vast diversity and abundance of microorganisms. The whole marine microbial communities including phyto and zooplankton, bacteria, archaea, unicellular eukaryotes, protozoans and fungi are estimated to account for more than 90% of the total aquatic biomass. These microorganisms are crucial to the survival of the higher organisms living in the oceans and other aquatic ecosystems that are highly dependent on the activities of complex marine microbial communities. Microorganisms can improve the water quality by naturally controlling the flux of nutrients, and also by degrading and recycling anthropogenic organic and inorganic contaminants. Moreover, imbalances in plankton microbial communities, usually caused by environmental shifts can compromise water quality and all associated uses. Hence, there is a great interest and need to study planktonic microbial communities on relevant temporal and spatial scales, to characterize their diversity and functional dynamics using the currently available highly sensitive genomic approaches.

The traditional sampling method of water planktonic implies the collection of determined volumes of water at a pre-determined depth, what for example in the ocean is traditionally done with Niskin bottles in an individual fashion or in a rosette configuration using an on-vessel crane. These sampling methods involve time and effort to collect and filter the water on board or at a home laboratory. This procedure also increases costs mainly due to the rental and operation of the vessel, and promotes deterioration of the sample, derived from the storage time until the filtration step. In addition, since the water needs to be extracted from the samplers and preserved on the vessel and/or in the lab until filtration, there is a risk of change of the physicochemical conditions of the water that can cause lysis of some microbial eukaryotes, also increasing the risk of potential contamination.

To date, few biosampler systems have been developed. Among the few biosampler systems available, a prototype for water filtration and sampling preservation of distinct biological class sizes was developed (Trembanis et al. 2012). However, this system is expensive to deploy, needs priori knowledge of the bio-life to be collected, requires high maintenance, and size limits its integration in smaller autonomous unmanned vehicles (AUV). A system to collect water through an AUV has been previously developed (Bird et al. 2007), but it is limited to small volumes of water and does not have the ability to concentrate water microplanktonic samples, limiting the use of those samples for some highly sensitive analytic genomic approaches. Some bio-samplers were also developed for in situ and real time detection of specific genetic targets using automated sampling and molecular techniques to enumerate the abundance of specific species and functional groups (e.g. McQuillan and Robidart, 2017; Scholin et al. 2009; Preston et al 2011). These systems are very powerful for some applications, but are extremely costly and limited to the identification of a particular protein, toxin and/or organism.

These facts are disclosed in order to illustrate the technical problem addressed by the present disclosure.

### GENERAL DESCRIPTION

The present disclosure relates to the development of a low cost in situ automatic bio-sampler device which allows collecting and concentrating, in particular by filtration, of water plankton samples to study the plankton microbiome, and could be easily connected to the AUV. Samples collected with the device now disclosed are suitable for highly sensitive analytic genomic approaches (genomic, metagenomic, and transcriptomic) to study the plankton microbiome, rather than specific species or functional group. The filtration efficiency and performance of the device were validated by comparison with conventional manual sample collection based on standard sampling and laboratory filtration protocols described in MicroB3 OSD Handbook (ten Hoopen et al. 2016), and by analyzing the reproducibility, eDNA recovery and diversity of prokaryotic (16S rDNA) and eukaryotic (18S rDNA) communities through massive sequencing analysis of samples collected by both filtering procedures.

Furthermore, the device now disclosed is a compact and low-cost autonomous biosampler, with the ability to yield DNA samples for later genomic analysis. This disclosure further demonstrates a similar performance between the device now disclosed and the standard manual protocol with respect to DNA recovery and microbiome diversity of Prokaryotic and microbial Eukaryotic communities at the abundant and rare members levels.

The device now disclosed is a small and compact system making it very convenient to transport. Also, the device now disclosed is very easy, simple to use and integrates a user-friendly application to program sampling definitions. The device now disclosed is a new resource for researchers interested in enhanced plankton microbial sampling; specially designed to be used, not only in oceanic research, but also in coastal, estuarine, riverine, lakes or aquaculture environments. The major advantage of the device now disclosed is allowing in situ filtrations of a large volume of water, increasing DNA yields and therefore, the possible detection of rare communities. The device now disclosed can be successfully employed to increase spatial and temporal resolution of aquatic microbiome monitoring. It will represent a key complement to fixed and mobile (e.g AUV) aquatic observation systems to tackle the biological knowledge gap in understudied remote aquatic ecosystems.

The present disclosure relates to a portable device for collecting and/or concentrating in situ plankton microbiome, configured for submersion in water, comprising:
an inlet for water containing the plankton microbiome;
an outlet for water deployed of plankton microbiome;
a plurality of valves between the inlet and the outlet;
a pump for pumping water from the inlet to the outlet such that water is passed across a filter cartridge;
a set of sensors for measuring flow and pressure;
the filter cartridge comprising a plurality of filters for in situ filtration of water containing the plankton microbiome;
an electronic control system with microcontroller for controlling the opening and closing of the plurality of valves and the speed of water pumping such that the device collects and/or concentrates in situ plankton microbiome.

The present disclosure further relates to a portable device for collecting and/or concentrating in situ plankton microbiome configured for submersion in water, comprising:
an inlet for water containing the plankton microbiome;
an outlet for water depleted of plankton microbiome;
a plurality of valves placed between the inlet and the outlet;
a set of sensors for measuring flow and pressure;
a pump for pumping water from the inlet to the outlet such that water is passed across a filter cartridge,
wherein the filter cartridge comprises a plurality of filters for in situ filtration of water containing plankton microbiome;
a microcontroller for controlling the opening and closing of a plurality of valves and the speed of water pumping such that the device collects and/or concentrates in situ plankton microbiome; a reservoir containing a preserving solution for preserving nucleic acids.

In an embodiment, the filter cartridge comprising a plurality of filters may be a filter cartridge comprising at least 16 filters with a pore size of 0.22 μm, although the filter pore size may change according with the sampling objective.

In an embodiment, the portable device may comprise at least 2 filter cartridges, preferably at least 4 filter cartridges, more preferably at least 8 filter cartridges.

In an embodiment, the portable device may further comprise a reservoir containing a preserving solution for DNA and/or RNA, wherein said preserving solution is for injection into the filter cartridge and as such preserve the DNA and/or RNA of the microbiome intact for long periods of time.

In an embodiment, the set of sensors may comprise a pressure sensor for controlling the pressure of the filter cartridge such that a pressure between 1-1.3 bar is reached.

In an embodiment, the set of sensors may comprise a flow sensor for detecting and/or controlling the flow of water that passes across the filter cartridge.

In an embodiment, the portable device now disclosed is for concentrating in situ plankton microbiome DNA by filtration.

In an embodiment, said portable device may operate at a depth of up to 150 m.

In an embodiment, the portable device may comprise a filter line for flushing and cleaning the device and as such avoid for example contaminations of the device.

In an embodiment, the plurality of valves may be a plurality of solenoid valves.

In an embodiment, the portable device may further comprise an electronic speed controller module for controlling the pump, preferably for controlling the motor of said pump.

In an embodiment, the portable device may further comprise a flow sensor for detecting the flow of the inlet and the flow of the outlet.

In an embodiment, the portable device may further comprise a valve manifold for flow distribution of water, preferably a manifold 1:6.

In an embodiment, the portable device may further comprise an analog pressure gauge for detecting the pressure of said device.

The present disclosure also applies to remote operated vehicle, autonomous underwater vehicle, a glider, a profiler, a submarine, a mini submarine, a human operated vehicle, a mooring, a buoy, a float or an off-shore station that comprises the portable device as described herein.

### BRIEF DESCRIPTION OF THE DRAWINGS

For an easier understanding of the disclosure, attached herein are figures which represent preferred embodiments of the disclosure that are not intended to limit the scope of protection of the present disclosure.

FIG. 1. Electronic, micro-hydraulic and filter components. System components such as pump, microcontroller, solenoid valve, flow sensor, manifold, pressure gauge, filters and filter cartridges used in the development of the device.

FIG. 2. System control architecture.

FIG. 3. System hydraulic diagram. Water and RNA/DNA storage reagent circuit with solenoid valves that control which circuit is being used and the relative location of the pump and sensors.

FIG. 4. Embedded microcontroller software structure. Representation of the five tasks that are running in the microcontroller and which sensor or actuator is being connected to it. The information between them passed though a set of message queues and global event bits.

FIG. 5. Embedded control software state machine. State machine implemented in the microcontroller firmware in the task State Machine. The flags “START”, “COMPLETE” and “ABORT” are set with information from the tasks that are processing the sensors signal.

FIG. 6. High level control and configuration. The user interface is based on a web page where the mission is configured and then saved on a local database (SSD disk). The mission in then passed to the embedded electronics through a RS-232 protocol.

FIG. 7. Filtration procedure design. The filtration of the different replicates started simultaneously in the OSD procedure and in the device in the different carboys.

FIG. 8. Field autonomous biosampler prototype components (left) and CAD model (right). The components are all mounted inside a cylinder in vacuum.

FIG. 9. Device prototype. Water inlet/outlet (A); external connector interface (B); opened in the field (C); integrated in a multi-sensor system.

FIG. 10. Filter cartridge box. Design of the filter cartridge box open (A) and closed (B); and Sterivex filter cartridge image (C).

FIG. 11. Examples of biosampler configuration and monitoring web pages. Two screenshots taken of the configuration web page. Top) Part of a configuration example of a water filtration mission. Bottom) Easy to read summary of the next mission to be executed.

FIG. 12. Laboratorial OSD filtration apparatus. (a) Diaphragm vacuum pump. (b) Water waste collection bottle. (c) PowerVac™ Manifold. (d) Sterivex filters. (e) 50 mL sterile syringes.

FIG. 13. Dendogram from the 16S rDNA (A) and 18S rDNA (B) at the Operational taxonomic units (OTUs) level. Dendogram generated from hierarchical analysis based on Bray-Curtis similarities of the lower triangular resemblance matrix obtained and using the Simprof test to verify significant differences (black and full lines) between clusters generated. Samples recovered using either the Ocean Sampling Day filtration standard procedure (OSD) or the device now disclosed (n=3). For the device now disclosed two filtration pressures were selected (1 and 1.3 bar).

FIG. 14. Dendogram from the rare (<1%) 16S rDNA (A) and 18S rDNA (B) at the Operational taxonomic units (OTUs) level. Generated from hierarchical analysis based on Bray-Curtis similarities of the lower triangular resemblance matrix obtained and using the Simprof test to verify significant differences (black and full lines) between clusters generated. Samples recovered using either the Ocean Sampling Day filtration standard procedure (OSD) or the device (n=3). For the device two filtration pressures (1 and 1.3 bar) were selected.

### DETAILED DESCRIPTION

One of the objectives of the portable device disclosed in the present subject-matter was to automate the process of water sampling collection and filtration for prokaryotic and eukaryotic microbiome analysis that is traditionally performed using manual procedures like in oceanographic and other aquatic ecosystems campaigns, such as the Ocean Sampling Day (OSD) (ten Hoopen et al. 2016[). This is intended to reduce the logistical and operational costs of biological studies in aquatic environments and to take advantage of current technologies to improve both the quality of data gathering and its efficiency.

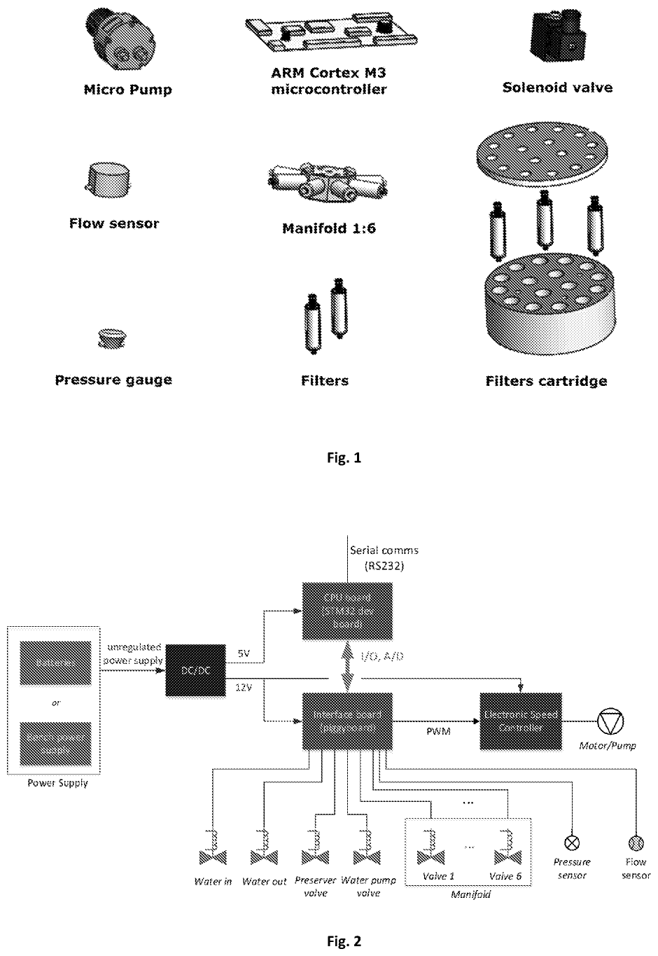

In an embodiment, the device comprises a set of electronic and micro-hydraulic components and circuits for in situ water sampling and filtering, comprising several components namely: a self-priming water pump (TCS MG2000), an ARM Cortex M4 microcontroller (STM32F411RE), a generic 100 A electronic speed controller (ESC) module, a flow sensor (Bio-Tech BT PCH-M-POM-LC 6), a Manifold 1:6 (NRESEARCH HP225T052), an analog pressure gauge (AVS-ROEMER E301), semi-rigid tubes for all wet circuits, push-in connections for all tubes, a set of filters and their cartridge (FIG. 1). The device was configured to use the same type of filters as those used in standard laboratory procedures (ten Hoopen et al. 2016), with a pore size of 0.22 μm.

In an embodiment, the device integrates full electronic control allowing for precise control and monitoring of the process. In addition, all the information on the performed sampling parameters and timestamp allows easy integration with data collected with other sensors. Embedded computer control is also relevant in order to integrate the device on autonomous systems such as AUVs.

In an embodiment, the architecture of the device is the one herein disclosed.

In an embodiment, the control and programming were implemented in a two-level hierarchical architecture (FIG. 2). A low-level microcontroller is responsible for the control of the micro-hydraulics circuit and related sensing. This device provides a set of functionalities that can be programmed/defined from a higher-level control computer.

In an embodiment, the control system for water filtration was based on the STM32F411RE ARM Cortex M4 microcontroller running a Real Time Operating System (FreeRTOS). The microcontroller receives the high-level mission definition through a RS232 communication line from a low power computer system. This computer system was based on an Odroid XU4 running Linux and has a set of databases which contains information of the tasks to be performed, as well as the status of the current filtering process and the logs of the previous filtering. This computer adjusts its clock via GPS when it is at surface and estimates the depth of the device using a pressure sensor. The microcontroller controls the opening and closing of the valves and the speed of the water pump.

In an embodiment, power supply can be provided externally (e.g. through an unregulated cabled DC source or by a lab bench power supply) or with an internal set of batteries. All the required regulated voltage lines for its components are produced in the device.

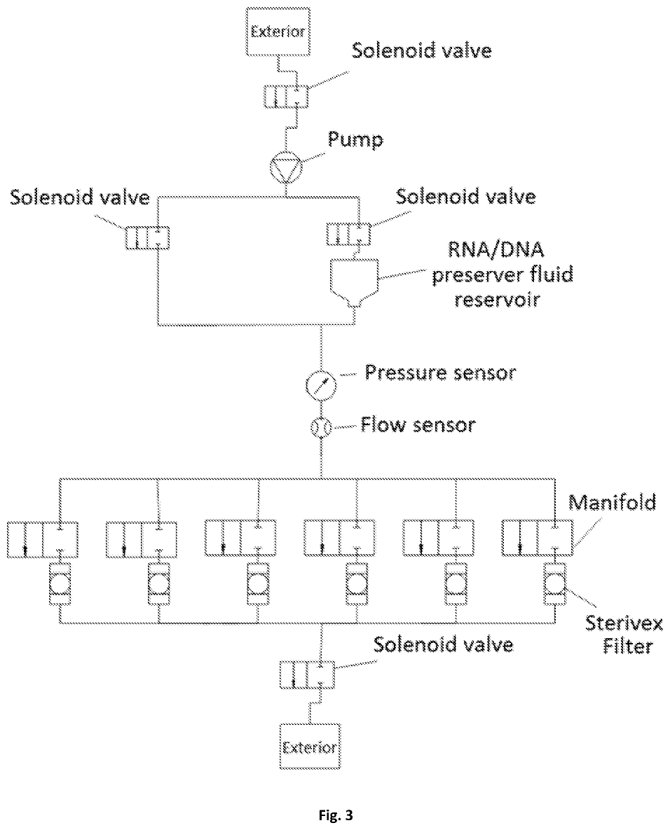

In an embodiment, the hydraulic circuit is represented in FIG. 3 (only one 6-filters manifold is exemplified). The water is pumped from the environment to one or more (replicates) sterile pressure driven filters with the pump controlled with an Electronic Speed Controller (ESC) via a Pulse-Width modulation signal (PWM), through the hydraulic circuit. These filters are selected by a set of valves arranged in the manifolds grouping six elements. Multiple manifolds can be used in order to select the desired number of available sample filters. After water filtration, the pump can inject into the filter a preserving DNA/RNA solution from an onboard reservoir. Pressure and flow sensors allow controlling both pressure and liquid flow to the filters (in both stages). An empty filter line (pass-through) is used to flush and clean the hydraulic circuit.

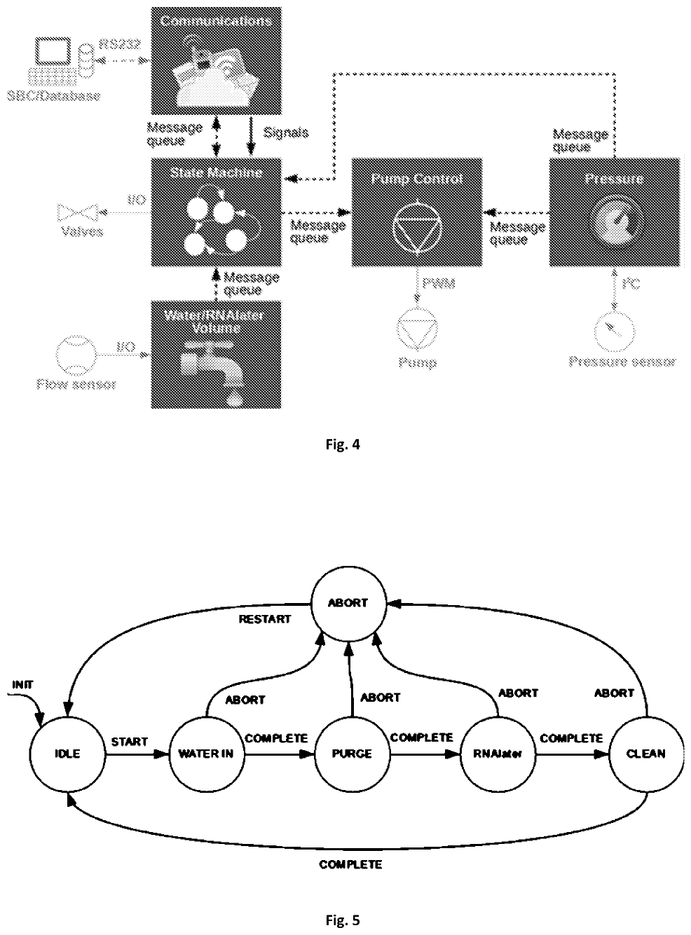

In an embodiment, the embedded firmware was based on the FreeRTOS (FIG. 4), a Real Time Operating System (RTOS) for the ARM Cortex M3, and starts by initializing all peripherals attached to the microcontroller. Peripherals include the pump, which has a PWM output, the valves that use an Input/Output (I/O), and the pressure sensor which has an analog output and is connected to the microcontroller 12-bit internal ADC and to the RS232 communication through the main board (FIG. 4).

In an embodiment, there are 5 tasks (or threads) running in the Real Time Operating System (communications, state machine, water/RNAlater volume, pump control and pressure). This implementation allows a simplified device to be developed and new features to be integrated since everything is contained in a separated task.

In an embodiment, the task Communications is responsible for reading the commands sent over RS232 by the main computer (SBC). These commands, after parsed, are passed into the correspondent task using the RTOS signals and/or message queues. The commands are mainly “START” or “STOP” the filtration process and the configuration parameters. The State Machine task implements the state machine described in FIG. 5. This task blocks until a START command arrives and, during its execution, receives sensor data via message queues from other tasks that are used to change its current state. The data that comes from the task Water/Preserved solution Volume calculates the volume of water filtered and the amount of preserved solution injected into the Sterivex filter after filtration. The output from the task that implements the state machine is sent to another task, Pump Control that is solely responsible for controlling the pump. This pump controlling task receives inputs from the task Pressure that reads the pressure sensor and processes its signals to obtain the pressure applied by the pump to the sterile pressure driven filters.

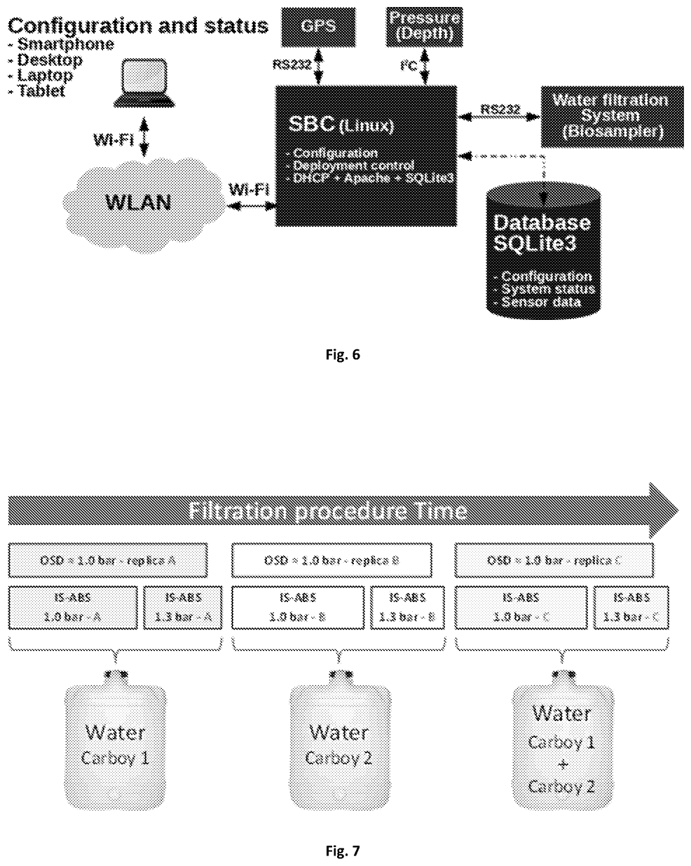

In an embodiment, an external environment pressure sensor allows estimating the depth and is available from the water filtration system electronics being its values obtained by the low power computer over I2C. The GPS is connected directly to the Single Board Computer (SBC) which synchronizes the clock using the Chrony service (FIG. 6). The SBC allows a flexible development and future integration of other sensors that may be required (e.g. saving a huge amount of data or having special communication protocols). Currently, the SBC provides a web interface based on PHP and SQLite3 using a Wi-Fi antenna that allows users to input the parameters to the filtering operation as well to monitor the current status of the biosampler (when it is at the surface). The mission can be configured by using any device with Wi-Fi and a web browser such as Smartphone, Desktop, Laptop or Tablet, allowing simple and fast setup of the filtration operation (FIG. 6).

In an embodiment, a filtration mission can be configured by the user by pre-setting a set of input parameters controlling the filtration process. These parameters include: (i) volume of water to be filtered; (ii) maximum pumping pressure; (iii) water column depth at which the filtration should start; (iv) number of simultaneous samples to be collected by filtration; (v) time of the day to start the filtration mission. The mission is configured by entering the number of sterile pressure driven filters available in the cartridge, the initial time of the sampling, the delay between collection of samples and how many replicates should be taken. This is done with a device with an internet browser that connects via Wi-Fi to the SBC. The SBC has a HTML server (Apache) with a configuration web page (FIG. 11) and saves every configuration provided by the user as well as the sensor data in a SQLite3 database.

In an embodiment, the configuration is then encapsulated by a service written for this purpose that runs in the operating system providing a simple interface for the user and also returning a feedback loop of the operation to be executed. The operation setup is then passed to the microcontroller via RS232 protocol.

In an embodiment, the tests of filtration volumes vs time performance were carried out as follows. The performance of the device in terms of filtration volumes and filtration time was assessed by monitoring the filtration of 2 L of water at three distinct constant working pressures (0.8, 1.3 and 1.8 bars). Filtration time was measured for each 100 ml of water filtered until a total filtration volume of 2 liters is obtained.

In an embodiment, the validation for microbiome analysis was carried out as follows. The prototype validation was performed by doing parallel filtration in the laboratory with the device and using a conventional OSD protocol (ten Hoopen et al. 2016). Thereafter, compare the results in terms of marine microbial diversity. Surface seawater samples were collected in November 2016 at approximately 25 km offshore, stored into two 20 L carboys and transported to the laboratory.

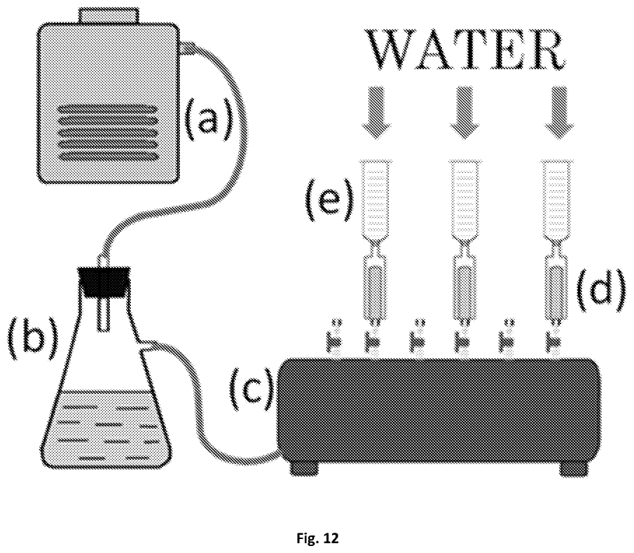

In an embodiment, the filtration procedures were carried out as follows. The OSD filtration apparatus (FIG. 12) consisted of a diaphragm vacuum pump (KNF N145 AN.18) linked to a water waste collection bottle which receives filtered water from 50 mL sterile syringes connected to a 0.22 μm sterile pressure driven filter. The vacuum pump has an ultimate vacuum of 100 mbar (abs), which creates a differential pressure of approximately 1 bar.

In an embodiment, the filtration procedures of the device utilized a peristaltic self-priming water pump (MG2000) and a 0.22 μm sterile pressure driven filter cartridge as.

In an embodiment, a total of 3 liters of coastal seawater were filtered in each sterile pressure driven filter. The comparison between laboratory standardized method and the device was carried out in triplicates (A, B, C) and at similar filtration pressure (≈1.0 bar). An additional filtration pressure (1.3 bars) was also tested for the device (FIG. 7). Sterile pressure driven filter units were stored at −80° C. until it is time for DNA extraction. DNA extraction is conducted following the OSD guidelines (ten Hoopen et al. 2016).

In an embodiment, to avoid potential differences between the two filtration procedures due to filtration time lapse and/or differences caused by seawater storage in different carboys, replicate filtrations started simultaneously in both procedures (FIG. 7). In addition, carboys were manually shaken immediately before each filtration to guarantee the homogeneity of the sample.

In an embodiment, the microbiome analysis was carried out as follows. DNA was extracted from each sterile pressure driven filter using DNA isolation kits following the manufacturer's instructions. Concentration and quality of DNA were measured by fluorometry. Environmental DNA obtained after extraction was used for 16S rDNA and 18S rDNA metabarcoding analysis targeting prokaryotes and eukaryotes, respectively. Hypervariable V4-V5 region (≈412 bp) of 16S rDNA gene was amplified using the universal primer pairs 515YF/Y906R-jed). For eukaryotes V4 region (≈434 bp) of 18S rDNA gene was amplified using TAReuk454FWD1/TAReukREV3_modified primers set. Paired-end sequencing was performed.

In an embodiment, the data analyses were carried out as follows. A comparative evaluation of microbial community structure detected by OSD manual procedure and the device was performed focusing on both total prokaryotic and eukaryotic communities and on the ‘rare biosphere’ (i.e. the pool of low-abundance taxa, threshold of 1%). Beta diversity of Prokaryotic and Eukaryotic communities were calculated using OTUs relative percentage values with PRIMER software (version 6.1.11).

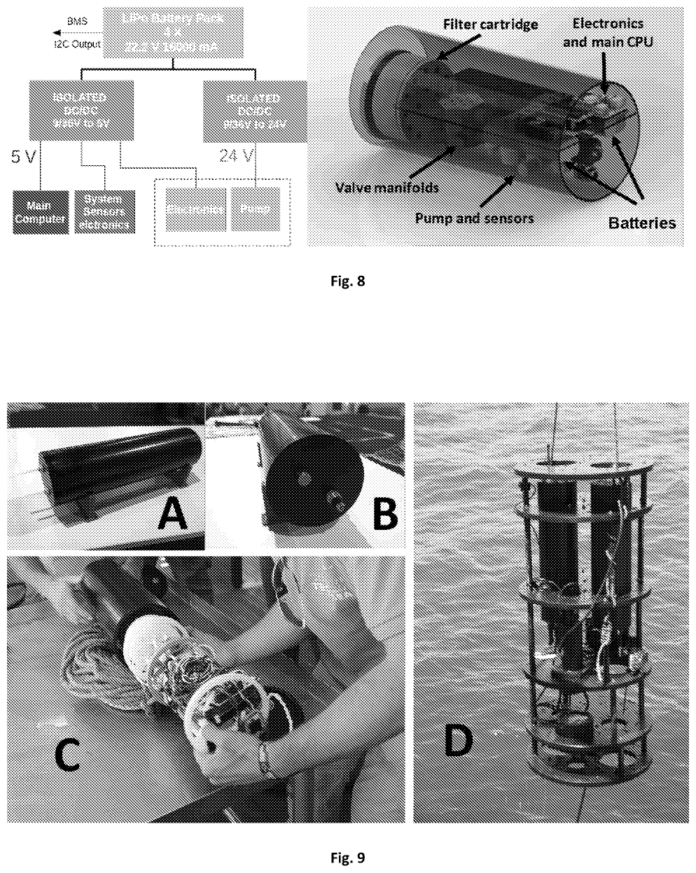

In an embodiment, the mechanical integration and functioning of the device may be as follows. The device includes the hydraulics components (FIG. 1A and FIG. 3) such as the water pump, microcontroller, ESC module, flow sensor, Manifold 1:6, analog pressure gauge, semi-rigid tubes for all wet circuits, push-in connections for all tubes, and a set of filters and their cartridge, embedded controller electronics, the main low power computer and a set of LiPo batteries (FIG. 8).

In an embodiment, the power source is based on a pack of 4 lithium ion polymer batteries with 22.2 V and 16000 mA with low weight and high density. These batteries are connected to two isolated wide input and low noise output DC/DC converters with 5V and 24 V outputs respectively. From this point every subsystem receives the necessary voltage input. For the electronic systems that need other voltages, such as 3.3 V, the voltages are provided in the printed circuit board by low dropout voltage regulators. The batteries are optional because the device can be integrated with other systems (for instance in a Remote Operated Vehicle or an Autonomous Underwater Vehicle) that can provide the necessary power.

In an embodiment, all the components were housed in a 150 mm diameter and 500 mm length aluminum pressure housing allowing for operation of up to 150 m depth (FIG. 9). For the hydraulic circuit, a set of flexible plastic tubes and fast connectors allowed for ease of maintenance and corrosion resistance. The standalone device now disclosed (FIGS. 9 A, B and C) has an external underwater connector (FIG. 9B) allowing for integration with other systems (FIG. 9D), such as multiple sensor system. The integration of the device now disclosed in different water observation systems (such as AUVs or fixed platforms) will dramatically increase the biological surveillance capabilities allowing the use of highly sensitive genomic approaches for the detection of the whole or specific microbial communities diversity and functions.

In an embodiment, the components of the hydraulic circuit, flexible plastic tubes and fast connectors are transparent and can be placed under UV light for sterilization and elimination of eventual DNA from exogenous microorganisms. Before the filtration procedure, these hydraulic circuit components can be easily set-up in the device.

In an embodiment, the device now disclosed operates as follows: firstly, in situ water from the intended location is pumped through the hydraulic circuit using a micropump (TCS MG2000) and then flushed throughout the device to clean eventual residues in the piping and valves. Thereafter, the filtration process starts and water is filtered in situ in one (controlled through the manifold system) or more (replicates) filters, in particular filters with a pore size of 0.22 μm, preferably sterile pressure driven filters placed in a filter cartridge. Preferably, the device has at least 16 filters a pore size of 0.22 μm (Sterivex filters). Filtering using multiple filters at the same time adds both redundancy and statistical significance to the data collected if one needs it for the metagenomics and metatranscriptomic analyses. This allows researchers to link the identity and activity of the microbiomes present in the water column with biological function at the exact time of sampling.

In an embodiment, the filtration process is controlled by the embedded control system according to the predefined parameters. Either the volume of water to be filtrated, the duration of the filtration process, or the detection of filter blocking can be used to end the process. Once the filtration ends, a DNA/RNA preserving solution is pumped into the filter to preserve the sample for posterior retrieval. Depending on the sampling and research requirements, the device can be expanded by adding groups of manifolds and filter cartridges to the prototype.

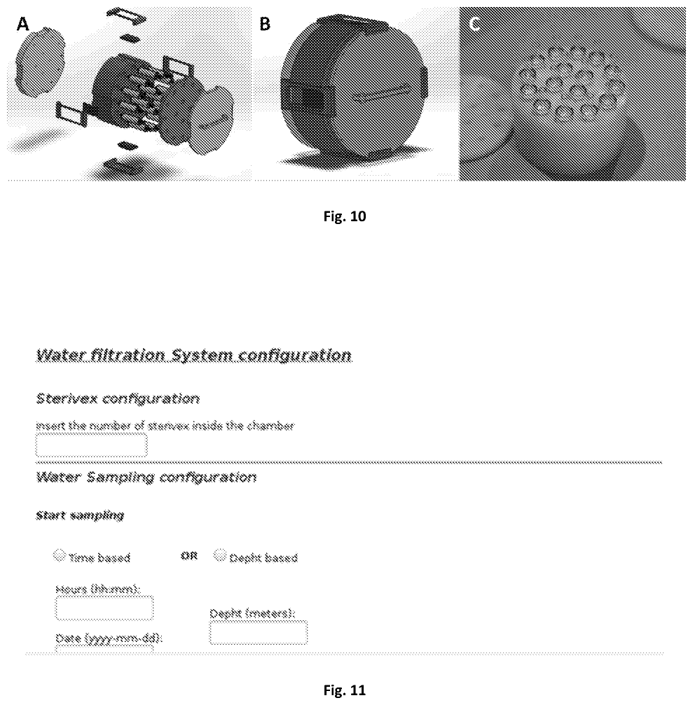

In an embodiment, the device now disclosed integrates a filter cartridge box made by, in particular, a set of pieces that can be coupled together (FIGS. 10A and B), and specially designed to easily store the cartridges with sterile pressure driven filters. Thus, the cartridge can conveniently be taken out of the device and sorted at the end of the filtration mission until DNA extraction. This box houses a set of filters, in particular 16 filters within the cartridge (FIG. 10) that can be removed individually or jointly, depending on the user's choice.

In an embodiment, these cartridge boxes were made of high-density polyethylene (HDPE) 1000 to maintain the properties of the cartridge at extreme temperatures. This also allows convenient storage of samples in cryogenic conditions, which is another suitable method to preserve samples until metagenomics and metatranscriptonics analyses. This allows long transport times (such as the ones occurring in a typical oceanographic campaign). Once in the lab, the individual sterile pressure driven filters can be removed for DNA/RNA extraction and sequencing.

In an embodiment, automated sampling devices capable of conducting eDNA sampling and molecular-biological sensing in situ are a promising approach for resolving high spatial and temporal water monitoring in different aquatic environments (McQuillan and Robidart et al. 2017). The device is capable of in situ water filtration, and of collection and preservation of microbiological material, with up to, preferably 16 sample filters per deployment, and in conditions compatible with subsequent metagenomic and metatranscriptomic studies. Moreover, it avoids DNA/RNA contaminations and biases related with management of water samples collected, since the device fixates the sample immediately after the filtration process. Also, the device now disclosed overcomes some limitations of the traditional Niskin bottle collections and shipboard filtration, such as bottle storage and transportation to home laboratory for filtration, reducing operational costs.

In an embodiment, the filtration flow performance may be as follows. The initial assessment of the device's filtration performance showed that increasing the pump speed (from 0.8 to 1.3 and to 1.8 bar) induced a higher average filtration flow and significantly lowered the filtration time considering the same volume (2 L) of water (Table 1).

TABLE 1
Filtration time and average flow. Water filtered, a total filtration
volume of 2 liters, and measured in fractions of 100 mL with the device
at 0.8, 1.0, and 1.3 bars (average ± standard deviation, n = 3).

| | Pressure: 0.8 bar | | Pressure: 1.3 bar | | Pressure: 1.8 bar | |
|---|---|---|---|---|---|---|
| | Time of | Average | Time of | Average | Time of | Average |
| Volume intervals | filtration | Flow | filtration | Flow | filtration | Flow |
| (mL) | (seconds) | (mL min⁻¹) | (seconds) | (mL min⁻¹) | (seconds | (mL min⁻¹) |
| 0-100 | 73 ± 1 | 82 ± 2 | 55 ± 1 | 110 ± 2 | 42 ± 2 | 144 ± 5 |
| 100-200 | 77 ± 1 | 77 ± 1 | 54 ± 1 | 112 ± 1 | 44 ± 2 | 138 ± 5 |
| 200-300 | 80 ± 2 | 75 ± 2 | 55 ± 1 | 109 ± 2 | 44 ± 1 | 137 ± 2 |
| 300-400 | 81 ± 2 | 74 ± 2 | 56 ± 1 | 107 ± 2 | 45 ± 2 | 133 ± 5 |
| 400-500 | 84 ± 1 | 71 ± 1 | 58 ± 1 | 104 ± 2 | 46 ± 2 | 131 ± 5 |
| 500-600 | 86 ± 2 | 70 ± 1 | 59 ± 1 | 101 ± 2 | 48 ± 3 | 124 ± 6 |
| 600-700 | 89 ± 0 | 68 ± 0 | 61 ± 1 | 99 ± 2 | 48 ± 3 | 126 ± 7 |
| 700-800 | 92 ± 0 | 65 ± 0 | 62 ± 1 | 97 ± 2 | 53 ± 2 | 114 ± 5 |
| 800-900 | 95 ± 1 | 63 ± 1 | 65 ± 2 | 93 ± 3 | 51 ± 1 | 118 ± 3 |
| 900-1000 | 98 ± 1 | 61 ± 1 | 67 ± 2 | 90 ± 3 | 54 ± 2 | 112 ± 3 |
| 1000-1100 | 101 ± 1 | 59 ± 1 | 69 ± 3 | 87 ± 4 | 56 ± 3 | 108 ± 6 |
| 1100-1200 | 105 ± 0 | 57 ± 0 | 71 ± 3 | 85 ± 3 | 57 ± 2 | 105 ± 4 |
| 1200-1300 | 112 ± 3 | 53 ± 1 | 73 ± 3 | 82 ± 4 | 59 ± 3 | 102 ± 6 |
| 1300-1400 | 117 ± 2 | 51 ± 1 | 76 ± 4 | 79 ± 4 | 63 ± 3 | 95 ± 4 |
| 1400-1500 | 122 ± 1 | 49 ± 1 | 79 ± 5 | 76 ± 5 | 66 ± 3 | 92 ± 5 |
| 1500-1600 | 128 ± 2 | 47 ± 1 | 84 ± 5 | 72 ± 5 | 69 ± 4 | 88 ± 5 |
| 1600-1700 | 135 ± 3 | 44 ± 1 | 88 ± 7 | 69 ± 5 | 72 ± 4 | 84 ± 5 |
| 1700-1800 | 147 ± 4 | 41 ± 1 | 91 ± 7 | 66 ± 5 | 76 ± 7 | 79 ± 7 |
| 1800-1900 | 156 ± 2 | 38 ± 0 | 96 ± 8 | 63 ± 5 | 82 ± 7 | 73 ± 7 |
| 1900-2000 | 168 ± 1 | 36 ± 0 | 102 ± 10 | 59 ± 6 | 87 ± 9 | 69 ± 7 |

Moreover, considering each filtration pressure tested, a significant (ANOVA, P<0.05) decrease in the average flow was recorded with increase of water volume filtrated or filtration time (Table 1). As compared with the manual procedure (35.8±0.3 min), filtration time substantially decreased (24±1 min) when equal volume of water (2 L) was filtered by the autonomous device (Table 1).

In an embodiment, results on the DNA recovered from the sterile pressure driven filters after filtering 3 liters of water at the same pressure (1 bar), using the standard OSD manual procedure and the device, showed a similar (P≥0.05) performance between these two methods (Table 2). The device had the advantage of having a lower time of filtration (Table 2) due to the higher average flow relative to the manual OSD procedure.

TABLE 2
Filtration time, volume, average flow and DNA recovered.
Table 2 shows the results of the tests performed with the
Ocean Sampling Day (OSD) standard procedure using the device
(mean ± standard deviation, n = 3). Two filtration
pressures were selected. Different superscript letters indicate
significant (ANOVA, P < 0.05) differences among the
three filtration procedures for each parameter.

| | OSD | Device now disclosed | |
|---|---|---|---|
| Pressure (bar) | ≈1 | 1 | 1.3 |
| Time of Filtration (minutes) | 128^{a} ± 16 | 61^{b} ± 4 | 56^{b} ± 5 |
| Mean Flux (mL/min) | 24^{a} ± 3 | 50^{b} ± 3 | 54^{b} ± 5 |
| DNA recovered (μg/mL) | 7^{a} ± 5 | 7^{a} ± 2 | 10^{a} ± 8 |
| Volume per replicate (L) | 3 | 3 | 3 |

Comparing the two pressures tested with the device now disclosed, no statistically significant differences (P>0.05) were observed, although at the higher pressure tested, an increase in variation (standard deviation) on the DNA recovered was observed (Table 2).

In an embodiment, the performance on sequences and OTUs recovered were performed as follows. DNA samples obtained from the different filtration tests, as explained above, were analyzed to explore prokaryotic (16S rDNA) and unicellular eukaryotic communities (18S rDNA) to highlight potentially different results in the community structure as a result of the manual and autonomous filtration procedures (OSD and device now disclosed). Moreover, a deeper comparison between samples filtered by the device now disclosed at 1 bar and 1.3 bars was also performed.

In an embodiment, a sorting procedure performed by Mothur pipeline v.1.38.1 produced a total curated dataset of 462956 (16S) and 227045 (18S) unique sequences. Clustering the reads at 97% of similarity for both prokaryotes and eukaryotes produced 385029 and 149725 OTUs (Table 3).

TABLE 3
Overview of the 16S and 18S datasets. Datasets were generated from OSD
standard methodologies and the device at 1 bar filtration pressure; and
with the device at two different filtration pressures (1 and 1.3 bar).
Different superscript letters indicate significant (ANOVA, P < 0.05)
differences among the three filtration procedures in each parameter.

| | | OSD | Device | |
|---|---|---|---|---|
| | | ≈1 bar | 1 bar | 1.3 bar |
| 16S rDNA | Raw paired-end | 78107^{a} ± 22162 | 53549^{a} ± 8106 | 48570^{a} ± 18049 |
| | Reads^{#} | | | |
| | Unique reads after | 63424^{a} ± 28551 | 47567^{a} ± 7453 | 43328^{a} ± 16119 |
| | filtering^{§} | | | |
| | OTUs clustered at 97%^{£} | 52369^{a} ± 19904 | 41085^{a} ± 5173 | 34889^{a} ± 10247 |
| 18S rDNA | Raw paired-end | 30177^{a} ± 20852 | 22510^{a} ± 8476 | 36019^{a} ± 27587 |
| | Reads^{#} | | | |
| | Unique reads after | 25776^{a} ± 17626 | 19195^{a} ± 7206 | 30710^{a} ± 23700 |
| | filtering^{§} | | | |
| | OTUs clustered at 97%^{£} | 18044^{a} ± 11812 | 13328^{a} ± 3921 | 18536^{a} ± 10160 |

^{#}Total number of paired-end sequences
^{§}Unique sequences left after quality control
^{£}OTUs obtained at 97% clustering after Metazoa and singletons removal

In an embodiment, the reproducibility of the filtration procedures on microbiome diversity was evaluated by comparing several diversity indices, including the number of observed OTUs, Chao1, Shannon, Berger Parker dominance, Simpson's evenness, and also the Good coverage (Table 4). General trends in diversity indices calculated showed no statistically significant (P>0.05) differences regardless of the filtration procedure tested (Table 4).

TABLE 4
Diversity indices for 16S and 18S rDNA. Table 4 shows the results of the quantity
of DNA recovered using either the Ocean Sampling Day filtration standard procedure
and the device (mean ± standard deviation, n = 3). Two filtration pressures
(1 and 1.3 bars) were used. Different superscript letters indicate significant (ANOVA,
P < 0.05) differences among the three filtration procedures for each diversity index.

| | | OSD | Device now disclosed | |
|---|---|---|---|---|
| | Diversity indices | ≈1 bar | 1 bar | 1.3 bar |
| 16S rDNA | Observed OTUs | 2523^{a} ± 417 | 2390^{a} ± 228 | 2650^{a} ± 462 |
| | Chao1 | 6370^{a} ± 2428 | 5589^{a} ± 253 | 7072^{a} ± 3096 |
| | Shannon index | 7.5^{a} ± 0.4 | 7.4^{a} ± 0.5 | 7.4^{a} ± 0.1 |
| | Berger Parker | 0.13^{a} ± 0.05 | 0.11^{a} ± 0.02 | 0.13^{a} ± 0.03 |
| | Simpson's evenness | 0.014^{a} ± 0.008 | 0.015^{a} ± 0.004 | 0.012^{a} ± 0.004 |
| | Good coverage | 0.94^{a} ± 0.02 | 0.94^{a} ± 0.01 | 0.93^{a} ± 0.02 |
| 18S rDNA | Observed OTUs | 583^{a} ± 220 | 648^{a} ± 60 | 625^{a} ± 77 |
| | Chao1 | 773^{a} ± 352 | 912^{a} ± 78 | 831^{a} ± 189 |
| | Shannon index | 7.0^{a} ± 0.2 | 6.8^{a} ± 0.4 | 6.7^{a} ± 0.4 |
| | Berger Parker | 0.07^{a} ± 0.01 | 0.10^{b} ± 0.02 | 0.13^{b} ± 0.05 |
| | Simpson's evenness | 0.09^{a} ± 0.04 | 0.06^{a} ± 0.01 | 0.06^{a} ± 0.04 |
| | Good coverage | 0.98^{a} ± 0.02 | 0.969^{a} ± 0.003 | 0.97^{a} ± 0.01 |

In an embodiment, the performance at high community taxonomy level was as follows. The occurrence of main archaea and bacteria phyla among samples recovered using either the OSD or the device filtration procedures showed similar (ANOVA, P≥0.05) relative percentage of OTUs within the different phyla analyzed (Table 5).

TABLE 5
Relative percentage (>1%) of 16S OTUs (Bacteria and Archaea) taxonomic composition
at phylum level. Table 5 shows the relative percentages of bacteria and archaea
detected in the tests performed with the Ocean Sampling Day (OSD) standard procedure
and with the device now disclosed (mean ± standard deviation, n = 3).
For the device now disclosed two filtration pressures were selected (1 and 1.3
bar). Different superscript letters indicate significant (ANOVA, P < 0.05)
differences among the three filtration procedures for each phylum.

| | | OSD | Device now disclosed | |
|---|---|---|---|---|
| | | ≈1 bar | 1 bar | 1.3 bar |
| Relative percentage | Alphaproteobacteria | 34^{a} ± 4 | 31^{a} ± 2 | 32^{a} ± 4 |
| of main Bacteria Phyla | Flavobacteriia | 29^{a} ± 2 | 32^{b} ± 1 | 30^{ab} ± 2 |
| | Gammaproteobacteria | 14^{a} ± 2 | 14^{a} ± 2 | 13^{a} ± 1 |
| | Cyanobacteria | 2.3^{a} ± 0.4 | 2.7^{ab} ± 0.5 | 2.9^{b} ± 0.2 |
| | Planctomycetacia | 2^{a} ± 1 | 2.6^{a} ± 0.4 | 2.7^{a} ± 0.7 |
| | Acidimicrobiia | 2.5^{a} ± 0.5 | 2.0^{a} ± 0.5 | 2.5^{a} ± 0.4 |
| | Sphingobacteriia | 2.4^{a} ± 0.4 | 2.5^{a} ± 1 | 2.0^{a} ± 0.1 |
| | Verrucomicrobiae | 1.9^{a} ± 0.2 | 1.7^{a} ± 0.4 | 1.9^{a} ± 0.5 |
| | Deltaproteobacteria | 1.5^{a} ± 0.1 | 1.3^{ab} ± 0.2 | 1.2^{b} ± 0.2 |
| | Betaproteobacteria | 0.6^{a} ± 0.4 | 2.4^{a} ± 3.2 | 1.9^{a} ± 2.3 |
| Relative percentage | Thaumarchaeota | 0.2^{a} ± 0.1 | 0.11^{a} ± 0.03 | 0.2^{a} ± 0.1 |
| of main Achaea Phyla | Woesearchaeota | 0.06^{a} ± 0.04 | 0.06^{a} ± 0.02 | 0.1^{a} ± 0.1 |
| | Euryarchaeota | 0.07^{a} ± 0.01 | 0.05^{a} ± 0.02 | 0.10^{a} ± 0.04 |
| | Diapherotrites | 0.004^{a} ± 0.002 | 0.005^{a} ± 0.006 | 0.002^{a} ± 0.002 |
| | Bathyarchaeota | 0.003^{a} ± 0.003 | 0.004^{a} ± 0.006 | 0.002^{a} ± 0.004 |
| | Archaea unclassified | 0.004^{a} ± 0.001 | 0.003^{a} ± 0.002 | 0.003^{a} ± 0.003 |
| | Lokiarchaeota | 0.001^{a} ± 0.001 | 0.001^{a} ± 0.001 | 0.002^{a} ± 0.002 |

The analysis of eukaryotic (18S rDNA) dominant taxa also showed statistically similar (ANOVA, P≥0.05) relative percentage of OTUs patterns between the OSD and the device now disclosed filtration procedures (Table 6).

TABLE 6
Relative percentage of 18S OTUs Taxonomic composition at phylum
level. Table 6 shows the relative percentage of 18S OTUs Taxonomic
composition at phylum level detected in the tests performed with
the Ocean Sampling Day (OSD) standard procedure and with the
device (mean ± standard deviation, n = 3). Two filtration
pressures were selected (1 and 1.3 bars). Different superscript
letters indicate significant (ANOVA, P < 0.05) differences
among the three filtration procedures for each phylum.

| | | OSD | Device now disclosed | |
|---|---|---|---|---|
| | | ≈1 bar | 1 bar | 1.3 bar |
| Relative | Alveolata | 36^{a} ± 2 | 37^{a} ± 3 | 36^{a} ± 2 |
| percentage of | Stramenopiles | 28^{a} ± 1 | 25^{b} ± 2 | 24^{ab} ± 4 |
| main 18S Phyla | Archaeplastida | 18^{a} ± 1 | 18^{a} ± 4 | 22^{a} ± 6 |
| | Opisthokonta | 10^{a} ± 3 | 12^{a} ± 6 | 11^{a} ± 3 |
| | Hacrobia | 3.6^{a} ± 0.4 | 3.2^{a} ± 0.2 | 3^{a} ± 1 |
| | Rhizaria | 2^{a} ± 1 | 4^{a} ± 2 | 1.7^{a} ± 0.3 |
| | Apusozoa | 0.8^{a} ± 0.5 | 0.8^{a} ± 0.3 | 0.6^{a} ± 0.3 |
| | Eukaryota | 0.5^{a} ± 0.4 | 0.5^{a} ± 0.4 | 0.6^{a} ± 0.5 |
| | unclassified | | | |
| | Amoebozoa | 0.4^{a} ± 0.3 | 0.6^{a} ± 0.2 | 0.5^{a} ± 0.1 |
| | Excavata | 0.1^{a} ± 0.1 | 0.2^{ab} ± 0.1 | 0.3^{b} ± 0.1 |

The results demonstrated that Prokaryotic and Eukaryotic taxonomic composition at higher levels was not affected by the two different filtration pressures applied in the device now disclosed (Tables 5 and 6).

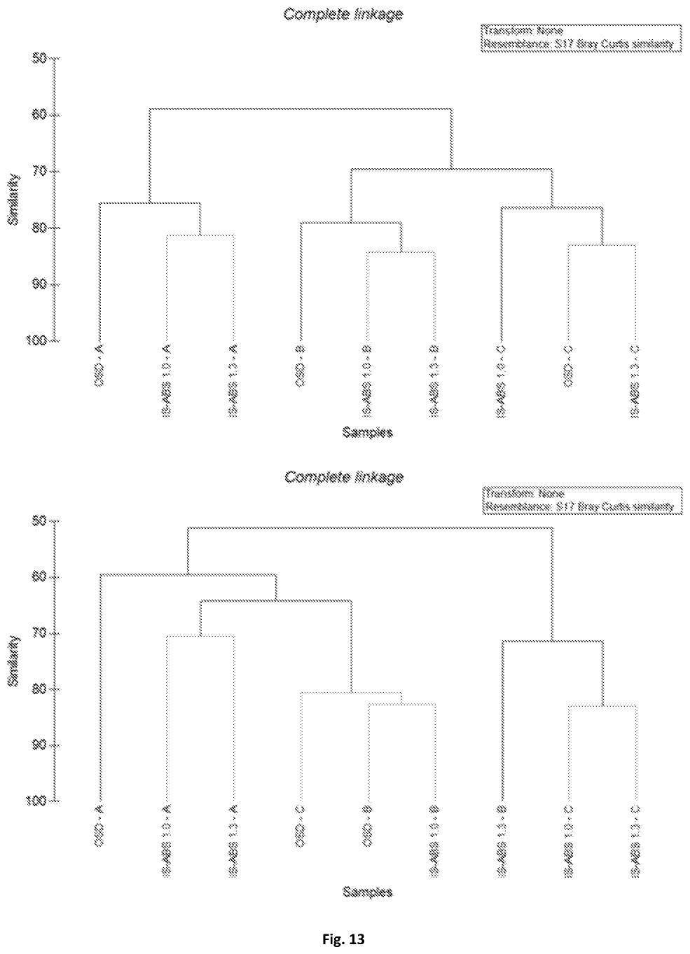

In an embodiment, the performance at community lower taxonomy level was as follows. A lower triangular resemblance matrix using Bray Curtis similarity was performed to identify potential effects of the different filtration procedures (OSD and device now disclosed). Prokaryotic (16S rDNA) community structure (FIG. 13A) at OTUs level showed dissimilarities among the replicates (A, B and C), indicating that the time lapse water filtration, and/or water from different carboy induced more differentiation (Simprof, P<0.05) than the type of filtration itself (OSD vs device).

In an embodiment, the results of the analyses at lower classification level did not show statistically significant (ANOVA, P≥0.05) differences in bacteria and archaea genera selected regardless of the filtration procedure used (Table 7).

TABLE 7
Distribution of the abundant taxa (>1%) retrieved from the 16S rDNA OTUs
taxonomic composition at lower taxonomic level. Table 7 shows the relative
percentage of 16S rDNA OTUs taxonomic composition at lower taxonomic level
detected in the testes performed with the Ocean Sampling Day (OSD) standard
procedure and with the device (mean ± standard deviation, n =
3). Two filtration pressures were selected (1 and 1.3 bar). No statistically
significant differences (ANOVA, P ≥ 0.05) were observed among the
three filtration procedures for the relative percentage of each genus.

| | OSD | | Device now disclosed | | | |
|---|---|---|---|---|---|---|
| | ≈1 bar | | 1 bar | | 1.3 bar | |
| Candidatus_Pelagibacter | 13.18 | ±4.97 | 9.42 | ±1.80 | 12.00 | ±4.11 |
| Tenacibaculum | 8.16 | ±1.20 | 9.89 | ±2.84 | 9.32 | ±1.94 |
| Flavobacteriales_unclassified | 2.20 | ±2.16 | 2.56 | ±2.55 | 2.49 | ±2.77 |
| Surface_2_ge | 3.48 | ±1.09 | 2.64 | ±0.42 | 3.14 | ±0.65 |
| Gammaproteobacteria_unclassified | 1.49 | ±1.23 | 1.62 | ±1.50 | 1.70 | ±1.58 |
| Erythrobacter | 1.34 | ±1.10 | 1.60 | ±1.16 | 1.53 | ±1.19 |
| Roseobacter_clade_NAC11-7_lineage | 1.90 | ±0.45 | 2.55 | ±0.94 | 2.21 | ±0.70 |
| Roseibacillus | 1.76 | ±0.20 | 1.55 | ±0.36 | 1.76 | ±0.45 |
| Rhodobacteraceae | 2.45 | ±0.58 | 3.15 | ±0.39 | 2.84 | ±0.17 |
| Flavobacteriaceae | 1.78 | ±0.05 | 1.86 | ±0.29 | 1.56 | ±0.10 |
| Prochlorococcus | 1.84 | ±0.35 | 2.13 | ±0.42 | 2.16 | ±0.07 |
| Hyphomonas | 0.98 | ±0.72 | 1.14 | ±0.94 | 1.02 | ±0.81 |
| Candidatus_Actinomarina | 1.27 | ±0.17 | 0.97 | ±0.16 | 1.26 | ±0.30 |
| Balneola | 0.60 | ±0.48 | 0.52 | ±0.25 | 0.56 | ±0.43 |
| Flavobacteriaceae | 1.61 | ±0.47 | 1.74 | ±0.56 | 1.83 | ±0.20 |
| Vibrio | 1.27 | ±0.25 | 1.42 | ±0.34 | 1.41 | ±0.58 |
| NS5_marine_group | 0.92 | ±0.05 | 0.88 | ±0.14 | 0.89 | ±0.14 |
| Planctomycetaceae_ uncultured | 0.45 | ±0.23 | 0.50 | ±0.12 | 0.59 | ±0.15 |

In an embodiment concerning the lowest taxonomic level of the eukaryotic community, results showed that samples from both filtration methods harbor both large (micro/mesoplankton) and small (picoplankton/nanoplankton) protists (Table 8). Indeed, when exploring the protistan community at lower taxonomic level it was identified, among the most abundant taxa (with a relative abundance higher than 1%), big cell size groups belonging to micro/mesoplankton: Bacillariophycae, Ciliophora and Dinophyceae such as Prorocentrum sp. (1% of abundance). Equally, has been recorded with the same abundance of 1% smaller photosynthetic groups e.g. the picoeukaryotes, MAST-8C_X_sp. Our data showed that all the genera are always present, independent of the filtration system used and pressures applied for both prokaryotic and eukaryotic communities.

TABLE 8
Distribution of the abundant taxa (>1%) retrieved from the 18S rDNA OTUs
taxonomic composition at lower taxonomic level. Table 8 shows the relative
percentage of 18S rDNA OTUs taxonomic composition at lower taxonomic level
detected in the testes performed with the Ocean Sampling Day (OSD) standard
procedure and with the autonomous biosampler (device now disclosed) (mean ±
standard deviation, n = 3). Two filtration pressures were selected (1 and
1.3 bar). No statistically significant differences (ANOVA, P ≥ 0.05) were
observed among the three filtration procedures for the relative percentage of each genus.

| | OSD | | Device now disclosed | | | |
|---|---|---|---|---|---|---|
| | ≈1 bar | | 1 bar | | 1.3 bar | |
| Prasino-Clade-VII-A_unclassified | 6.51 | ±1.27 | 8.99 | ±1.91 | 13.26 | ±5.57 |
| Labyrinthulaceae_X_sp. | 5.37 | ±1.91 | 6.79 | ±2.12 | 4.69 | ±1.92 |
| Aspergillus_clavatus | 4.90 | ±0.77 | 6.07 | ±4.14 | 5.57 | ±3.72 |
| Bathycoccus_prasinos | 6.15 | ±1.37 | 3.96 | ±1.09 | 2.76 | ±1.26 |
| Dino-Group-I-Clade-1_X_sp. | 2.86 | ±1.47 | 4.28 | ±1.05 | 4.75 | ±2.28 |
| Uncultured_Lecanicillium | 3.45 | ±2.12 | 3.78 | ±2.02 | 3.91 | ±0.96 |
| Dino-Group-I-Clade-1_X_sp. | 3.11 | ±0.68 | 4.01 | ±0.37 | 3.97 | ±0.99 |
| Thalassiosira_tenera | 3.66 | ±0.31 | 2.76 | ±0.70 | 2.88 | ±0.23 |
| Dino-Group-I-Clade-1_X_sp._strain8 | 1.77 | ±0.09 | 2.12 | ±0.43 | 1.42 | ±0.31 |
| Oxytricha_saltans | 1.34 | ±0.41 | 1.06 | ±0.13 | 1.87 | ±1.53 |
| Dino-Group-I-Clade-5_X_sp. | 1.07 | ±0.03 | 1.30 | ±0.26 | 0.85 | ±0.13 |
| Prorocentrum_sp. | 1.11 | ±0.18 | 1.08 | ±0.31 | 0.96 | ±0.04 |
| Paracineta_limbata | 0.92 | ±0.80 | 1.20 | ±0.87 | 0.84 | ±0.19 |
| MAST-8C_X_sp. | 1.33 | ±0.44 | 0.84 | ±0.17 | 0.69 | ±0.40 |

The results showed no statistically significant differences in prokaryotic and eukaryotic communities (not significant) at lower taxonomic composition level induced by different filtration procedures (device now disclosed and standard OSD).

In an embodiment, prokaryotic and eukaryotic rare species (<1% relative abundance) are increasingly recognized as crucial since they can have an over-proportional role in biogeochemical cycles and may be a hidden driver of microbiome function, such as in the response to organic pollutants. An overview of rare OTUs clustered at 97% (Table 9) revealed no statistically significant (ANOVA, P≥0.05) differences regardless of the different filtrations systems (OSD and device) used.

TABLE 9
The number the rare (<1%) OTUs (97%) in the 16S and 18S rDNA. Table 9 shows the
quantity of DNA obtained from the different procedures (Ocean Sampling day (OSD)
and in situ autonomous filtration prototype (device now disclosed); and different
filtration pressures (1 and 1.3 bar). Information for each treatment replicates
(A, B and C) and for total samples. Raw read pairs directly obtained from DNA-to-
data (for example Illumina MiSeq) sequencing platform, the sequence count after
cleaning by mothur analysis pipeline, for each group. The different superscript
letters show significant (ANOVA, P < 0.05) differences among filtration procedures.

| | | OSD | Device now disclosed | |
|---|---|---|---|---|
| | Pressure | ≈1 bar | 1 bar | 1.3 bar |
| 16S rDNA | OTUs clustered at 97% | 26500^{a} ± 7880 | 20594^{a} ± 1445 | 17068^{a} ± 4587 |
| 18S rDNA | OTUs clustered at 97% | 10425^{a} ± 6430 | 7246^{a} ± 2468 | 9939^{a} ± 5324 |

In an embodiment, the reproducibility and effects of the filtration procedures on rare (<1%) microbiome diversity were evaluated by comparing several diversity indices, including the number of observed OTUs, Chao1, Shannon, Berger Parker dominance, Simpson's evenness, and also the Good coverage (Table 10). Trends in the diversity indices showed no significant (ANOVA, P≥0.05) differences regardless the filtration procedure.

TABLE 10
Diversity indices for rare (<1%) 16S and 18S rDNA. Table 10 shows the DNA
detected in the tests performed with the Ocean Sampling Day (OSD) standard
procedure and with the autonomous DNA sampler (device now disclosed)
(mean ± standard deviation, n = 3). Two filtration pressures were
selected (1 and 1.3 bar). Different superscript letters indicate significant
(ANOVA, P < 0.05) differences among the three filtration procedures for
each diversity index.

| | | OSD | Device now disclosed | |
|---|---|---|---|---|
| | Diversity indices | ≈1 bar | 1 bar | 1.3 bar |
| 16S rDNA | Observed OTUs | 2531^{a} ± 558 | 2375^{a} ± 132 | 2680^{a} ± 516 |
| | Chao1 | 6338^{a} ± 2789 | 5494^{a} ± 637 | 7202^{a} ± 3218 |
| | Shannon index | 9.5^{a} ± 0.2 | 9.3^{a} ± 0.3 | 9.5^{a} ± 0.3 |
| | Berger Parker | 0.024^{a} ± 0.002 | 0.03^{a} ± 0.01 | 0.03^{a} ± 0.01 |
| | Simpson's evenness | 0.10^{a} ± 0.02 | 0.09^{a} ± 0.03 | 0.09^{a} ± 0.00 |
| | Good coverage | 0.87^{a} ± 0.05 | 0.88^{a} ± 0.01 | 0.86^{a} ± 0.04 |
| 18S rDNA | Observed OTUs | 585^{a} ± 236 | 674^{a} ± 49 | 639^{a} ± 106 |
| | Chao1 | 828^{a} ± 430 | 944^{a} ± 80 | 841^{a} ± 209 |
| | Shannon index | 7.9^{a} ± 0.5 | 8.0^{a} ± 0.3 | 8.0^{a} ± 0.2 |
| | Berger Parker | 0.025^{a} ± 0.005 | 0.05^{a} ± 0.04 | 0.03^{a} ± 0.01 |
| | Simpson's evenness | 0.26^{a} ± 0.07 | 0.18^{a} ± 0.06 | 0.22^{a} ± 0.04 |
| | Good coverage | 0.95^{a} ± 0.03 | 0.94^{a} ± 0.01 | 0.95^{a} ± 0.02 |

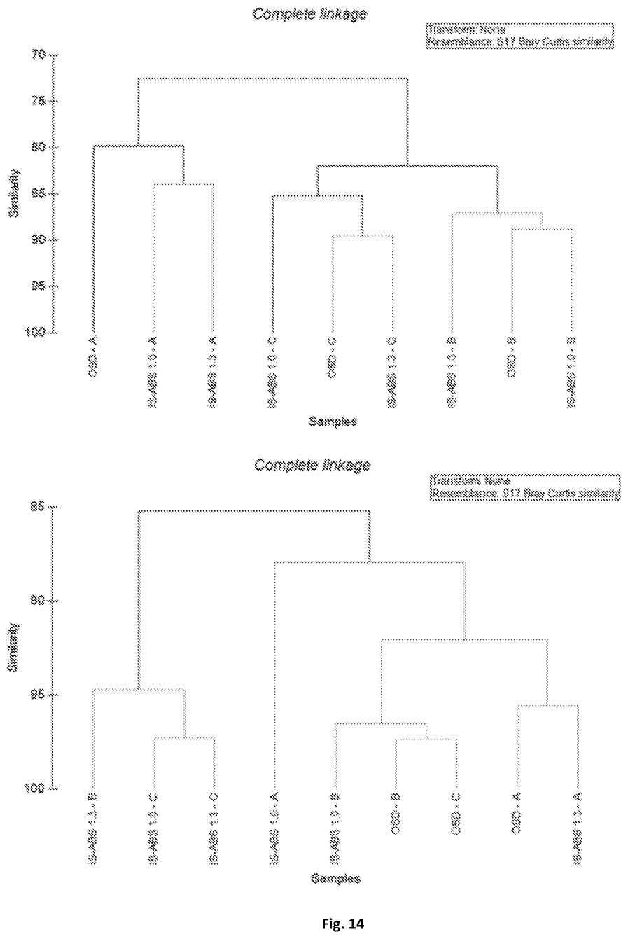

In an embodiment, at OTUs level (lower taxonomic level), the lower triangular resemblance matrix showed that rare (<1%) prokaryotic (16S rDNA) community (FIG. 14A), and eukaryotic (18S rDNA) community (FIG. 14B) did not show major differences regardless of the different filtration systems (OSD and device). Thus, the device is capable of detecting shifts in low relative abundant (<1%) microplankton groups.

The disclosure should not be seen in any way restricted to the embodiments described and a person with ordinary skills in the art will foresee many possibilities to modifications thereof.

Furthermore, where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the disclosure, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The above described embodiments are combinable. The following claims further set out particular embodiments of the disclosure.

### REFERENCES

1. Trembanis, A. C., Cary, C., Schmidt, V., Clarke, D., Crees, T., & Jackson, E. (2012, October). Modular autonomous biosampler (MAB)—A prototype system for distinct biological size-class sampling and preservation. In Oceans, 2012 (pp. 1-6). IEEE.
2. Bird L. E., Sherman A., Ryan J. 2007. Development of an Active, Large Volume, Discrete Seawater For autonomous Underwater Vehicles. Monterey Bay Aquarium Research Institute [0-933957-35-1 2007 MTS]
3. McQuillan, J. S., & Robidart, J. C. (2017). Molecular-biological sensing in aquatic environments: recent developments and emerging capabilities. Current opinion in biotechnology, 45, 43-50.
4. Scholin, C., G. Doucette, S. Jensen, B. Roman, D. Pargett, R. Marin III, et al. 2009. Remote detection of marine microbes, small invertebrates, harmful algae, and biotoxins using the Environmental Sample Processor (ESP). Oceanography 22(2):158-167
5. Preston et al. 2011 Preston C M, et al. (2011) Underwater application of quantitative PCR on an ocean mooring. PLoS One 6(8):e22522.
6. ten Hoopen, P., Cochrane, G., Schaap, D., Kottmann, R., Broggiato, A., von Kries, C., et al. 2016. Ocean Sampling Day Handbook.
7. Ribeiro, H., de Sousa, T., Santos, J. P., Sousa, A. G., Teixeira, C., Monteiro, M. R., et al. (2018). Potential of dissimilatory nitrate reduction pathways in polycyclic aromatic hydrocarbon degradation. Chemosphere, 199, 54-67.

1. A portable device for collecting and/or concentrating in situ plankton microbiome configured for submersion in water, comprising:
an inlet for water containing the plankton microbiome;
an outlet for water depleted of plankton microbiome;
a plurality of valves placed between the inlet and the outlet;
a set of sensors for measuring flow and pressure;
a pump for pumping water from the inlet to the outlet such that water is passed across a filter cartridge,
wherein the filter cartridge comprises a plurality of filters for in situ filtration of water containing plankton microbiome;
a microcontroller for controlling the opening and closing of a plurality of valves and the speed of water pumping such that the device collects and/or concentrates in situ plankton microbiome;
a reservoir containing a preserving solution for preserving nucleic acids.

2. The device according to claim 1, wherein the filter cartridge comprises at least 16 filters each having a pore size of 0.22 μm.

3. The device according to claim 1, comprising at least 2 filter cartridges.

4. The device according to claim 1, wherein the preserving solution is injectable into the filter cartridge.

5. The device according to claim 1, wherein the set of sensors comprises a pressure sensor for controlling and maintaining the pressure of the filter cartridge from 1 bar to 1.3 bar.

6. The device according to claim 1, wherein the set of sensors comprises a flow sensor for detecting and/or controlling the flow of water passing through the filter cartridge.

7. The device according to claim 1, for concentrating in situ plankton microbiome nucleic acids by filtration.

8. The device according to claim 1, wherein said device operates at a depth up to 150 m.

9. The device according to claim 1, further comprising a filter line for flushing and cleaning the device.

10. The device according to claim 1, wherein the plurality of valves is a plurality of solenoid valves.

11. The device according to claim 1, further comprising an electronic speed controller module for controlling the pump.

12. The device according to claim 1, further comprising a flow sensor for detecting the flow of solution through the inlet and the outlet.

13. The device according to claim 1, further comprising a valve manifold for flow distribution of water.

14. The device according to claim 1, further comprising an analog pressure gauge for detecting the pressure of said device.

15. An apparatus comprising the device according to claim 1, wherein the apparatus is a remote operated vehicle, an autonomous underwater vehicle, a glider, a profiler, a submarine, a mini submarine, a human operated vehicle, a mooring, a buoy, a float or an off-shore station.

16. The device according to claim 3, comprising at least 4 filter cartridges.

17. The device according to claim 16, comprising at least 8 filter cartridges.

18. The device according to claim 11, wherein the electronic speed controller module is for controlling a motor of said pump.
